(19) United States
(12) Patent Application Publication
(10) Pub. No.: US 2025/0258980 A1
(43) Pub. Date: Aug. 14, 2025

(54) ARTIFICIAL INTELLIGENCE-BASED DEVICE, METHOD, AND PROGRAM FOR PREDICTING PHYSICAL PROPERTIES OF MIXTURES

(71) Applicants: LG MANAGEMENT DEVELOPMENT INSTITUTE CO., LTD., Seoul (KR); LG ENERGY SOLOTION, LTD., Seoul (KR)

(72) Inventors: Changyoung PARK, Seoul (KR); Hongjun YANG, Seoul (KR); Jaewan LEE, Seoul (KR); Sehui HAN, Seoul (KR); Hyelim JEON, Daejeon (KR); Boram LEE, Daejeon (KR); Changhoon LEE, Daejeon (KR)

(21) Appl. No.: 19/183,509

(22) Filed: Apr. 18, 2025

Related U.S. Application Data
(63) Continuation of application No. PCT/KR2023/016080, filed on Oct. 17, 2023.

(30) Foreign Application Priority Data
Oct. 18, 2022 (KR) 10-2022-0134293

(51) Int. Cl. G06F 30/27; G16C 20/30; G16C 20/70

(52) U.S. Cl. CPC G06F 30/27; G16C 20/30; G16C 20/70

(57) ABSTRACT

A device for predicting the physical properties of a mixture including a plurality of component materials is disclosed. The device may comprise a memory in which a first AI model trained to output first feature data of material information, and a second AI model trained to output physical property prediction information of the first feature data are stored, and a processor for executing the first AI model and the second AI model, wherein the processor may input, into the first AI model, material information of each of the plurality of materials to acquire first feature data of each of the plurality of materials, and input the first feature data into the second AI model to acquire physical property prediction information of the mixture.

### CROSS REFERENCE TO RELATED APPLICATIONS

This application is a Bypass Continuation of International Patent Application No. PCT/KR2023/016080, filed on Oct. 17, 2023, which claims priority from and the benefit of Korean Patent Application No. 10-2022-0134293, filed on Oct. 18, 2022, each of which is hereby incorporated by reference for all purposes as if fully set forth herein.

### FIELD OF THE INVENTION

The present invention provides an artificial intelligence (AI)-based device, method and program for predicting the physical properties of a homogeneous mixture based on material aspects of its components. More particularly, the invention is directed toward sequential use of a first AI model trained to output first feature data and a second AI model trained to convert the first feature data into information that can be interpreted as a physical property prediction for the mixture.

### BACKGROUND OF THE INVENTION

Recently, as consumer demands and demand trends have diversified and product development methods have diversified, the need for new mixtures that can be used in product manufacturing has increased, and such new mixtures are being actively pursued. In order to more efficiently develop and mass-produce a new mixture that can be used in product manufacturing, researchers often find that reliable prediction of the physical properties of candidate mixtures is essential.

Conventionally, non-AI technologies for predicting or analyzing physical properties for a single material or an alloy have been developed and utilized. However, as the number of materials constituting target mixtures increases and the range of possible component ratios become more complex and extensive, efficient and accurate prediction of the physical properties of the target mixture becomes more and more difficult using these conventional technologies. Accordingly, there is an increasing need for a more efficient method for predicting the physical properties of a mixture.

The above information disclosed in this Background section is provided to aid in understanding the inventive concepts and is not admitted to be or to describe prior art with respect to the present invention.

### SUMMARY OF THE INVENTION

It is to be understood that both the foregoing general description of the invention and the following detailed description are exemplary, and thus do not restrict the scope of the invention.

The present disclosure is directed to providing an artificial intelligence-based device, method, and program for predicting the physical properties of a mixture based on material aspects of its components.

Objects of the present disclosure are not limited to the above-described object, and other objects that are not mentioned will be clearly understood by those skilled in the art from the following description.

According to an aspect of the present disclosure, there is provided a device for predicting physical properties of a mixture including a plurality of component materials, which device may include a memory storing a first artificial intelligence (AI) model trained to output first feature data associated with material information corresponding to specific component materials and a second AI model trained to output mixture physical property prediction information associated with the first feature data, and a processor configured to execute the first AI model and the second AI model, wherein the processor may be configured to obtain first feature data associated with each of the plurality of component materials by inputting, into the first AI model, material information corresponding to each of the plurality of component materials, and obtain mixture physical property prediction information by inputting the first feature data into the second AI model.

In some embodiments, he first AI model can include an attention-based model, and the attention-based model can be trained to extract feature data of a specific component material based on at least one of polarizability and hydrophobicity of the specific component material.

In some embodiments, the first AI model can include a molecular contrastive learning-based model, and the molecular contrastive learning-based model can be trained to align molecules with similar structures on a latent space.

In some embodiments, the first AI model can be trained to update second feature data extracted from two-dimensional graph information of a specific component material based on third feature data extracted from three-dimensional structural characteristics of the specific component material, and can be trained to output the first feature data based on the updated data.

In some embodiments, the three-dimensional structural characteristics of the specific component material can include a plurality of spatial arrangement conformers of the specific component material.

In some embodiments, the second AI model can include a multi-layer perceptron-based model.

In some embodiments, the second AI model can include a transformer encoder model.

In some embodiments, the processor can be configured to obtain physical property prediction information associated with the mixture by inputting the first feature data and component ratio information of the plurality of component materials in the mixture into the second AI model.

In some embodiments, the first AI model and the second AI model can be trained in an end-to-end training manner.

In some embodiments, the material information can include chemical information, and the chemical information can include molecular information of each of the plurality of component materials. Molecular information can take the form of a simplified molecular-input line-entry system (SMILES), an international chemical identifier (INCHI), or a self-referencing embedded string (SELFIES)) of each of the plurality of component materials.

In some embodiments, the chemical information can include at least one of atom properties and bond properties related to the mixture. In certain embodiments, at least one of the atomic property and the bond property related to the mixture can be obtained based on the molecular information.

In some embodiments, the first feature data can include molecular feature data of each of the plurality of component materials.

In some embodiments, the physical property prediction information can include Gibbs free energy prediction information.

In another aspect, the present invention provides a method for predicting physical properties of a mixture including a plurality of component materials, performed by a device, wherein the method can include obtaining first feature data associated with each of the plurality of component materials by inputting material information associated with each of the plurality of component materials into a first AI model that has trained first feature data associated with material information, and obtaining physical property prediction information associated with the mixture by inputting the first feature data into a second AI model that has trained physical property prediction information associated with the first feature data.

In some embodiments, the material information can be at least one of a simplified molecular-input line-entry system (SMILES), an international chemical identifier (INCHI) and a self-referencing embedded string (SELFIES). In some embodiments, the material information can be a polarizability or a hydrophobicity.

In some embodiments, a computer program that is stored in a computer-readable recording medium coupled with a hardware device to execute the method for predicting the physical properties of the mixture including the plurality of component materials can be further provided.

In some embodiments, a computer-readable recording medium that records a computer program for implementing methods of the present disclosure can be further provided.

According to the above configuration of the present disclosure, an artificial intelligence-based device, method, and program for predicting physical properties of a mixture can be provided.

In addition, according to the above configuration of the present disclosure, the physical properties of the mixture can be more efficiently predicted by using an artificial intelligence model that can consider interactions between components of the mixture.

Effects of the present disclosure are not limited to the above effects, and other effects that are not mentioned will be clearly understood by those skilled in the art from the following description.

Additional features of the inventive concepts will be set forth in the description which follows, and in part will be apparent from the description, or may be learned by practice of the inventive concepts.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts. The drawings are for illustration purposes only and are not intended to limit the scope of the present teachings in any way.

FIG. 1 is a schematic diagram of a system for implementing an artificial intelligence-based method for predicting physical properties of a mixture according to one embodiment of the present disclosure.

FIG. 2 is a block diagram for describing a configuration of a device that performs an artificial intelligence-based method for predicting the physical properties of the mixture according to one embodiment of the present disclosure.

FIG. 3 is a block diagram for describing an artificial intelligence-based method for predicting the physical properties of the mixture according to one embodiment of the present disclosure.

FIG. 4 is a block diagram for describing a configuration and operation of an artificial intelligence (AI) model for predicting the physical properties of a mixture according to one embodiment of the present disclosure.

FIG. 5 is a diagram for describing a training/inference method of an AI model for predicting the physical properties of a mixture according to one embodiment of the present disclosure.

FIGS. 6A and 6B are diagrams for describing a training method of an AI model that extracts feature data of materials constituting a mixture according to one embodiment of the present disclosure.

FIGS. 7A, 7B, and 7C are diagrams for describing type-specific performance of AI models according to one embodiment of the present disclosure.

FIGS. 8A, 8B, and 8C are diagrams for describing structures of AI models for predicting the physical properties of the mixture according to one embodiment of the present disclosure.

FIGS. 9A, 9B, and 9C are diagrams for describing training/inference methods of AI models for predicting the physical properties of the mixture according to one embodiment of the present disclosure.

### DETAILED DESCRIPTION

Exploring the expected physical properties of hypothetical new chemical mixtures is very important in many areas of science and engineering, such as fine chemicals production and use, chemical engineering, environmental science, food science, pharmaceuticals and tribology, to name a few. Modern researchers find that experimentally investigating all mixtures of interest is not in any way practical, and the use of computers to execute AI methods in order to characterize mixtures anticipated to be potentially useful is likely to soon become essential for investigators who wish to remain competitive in their respective fields.

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

The same reference numerals refer to the same components throughout the present disclosure. The present disclosure does not describe all elements of the embodiments, and common content in the art to which the present disclosure pertains or content that overlaps between the embodiments may be omitted. Terms “unit,” “module,” “member,” and “block” used in the specification may be implemented as software or hardware, and according to the embodiments, a plurality of “units,” “modules,” “members,” and “blocks” may be implemented as one component, or one “unit,” “module,” “member,” and “block” may also include a plurality of components.

Throughout the specification, when a first component is described as being “connected” to a second component, this includes not only a case in which the first component is directly connected to the second component but also a case in which the first component is indirectly connected to the second component, and the indirect connection includes connection through a wireless communication network.

In addition, when a certain portion is described as “including,” a certain component, this means further including other components rather than precluding other components unless specifically stated otherwise.

In each operation, identification symbols are used for convenience of description, and the identification symbols do not describe the sequence of each operation, and each operation may be performed in a different sequence from the specified sequence unless a specific sequence is clearly described in context.

Hereinafter, the operation principles and embodiments of the present disclosure will be described with reference to the accompanying drawings.

‘A device for predicting physical properties of a mixture including two or more types of materials according to the present disclosure’ in the present specification includes all of various devices that can perform computational processing and provide results to a user. For example, the device for predicting the physical properties of the mixture including two or more types of materials according to the present disclosure may include all of a computer, a server device, and a portable terminal, or can be in a form of any one or a combination including a plurality of them.

Here, the computer can include, for example, a notebook, a desktop, a laptop, a tablet PC, a slate PC, etc., which are equipped with a web browser.

The server device is a server that processes information in communication with an external device, and may include an application server, a computing server, a database server, a file server, a game server, a mail server, a proxy server, and a web server.

The portable terminal is, for example, a wireless communication device ensuring portability and mobility and can include all kinds of handheld-based wireless communication devices such as a personal communication system (PCS), a global system for mobile communications (GSM), a personal digital cellular (PDC), a personal handyphone system (PHS), a personal digital assistant (PDA), international mobile telecommunication-2000 (IMT-2000), code division multiple access-2000 (CDMA-2000), w-code division multiple access (W-CDMA), a wireless broadband internet (WiBro) terminal, a smart phone, and wearable devices such as a watch, a ring, a bracelet, an anklet, a necklace, glasses, contact lenses, or a head-mounted device (HMD).

In addition, in describing the present disclosure, the mixture can include an electrolyte of a battery, but is not limited thereto, and can mean various types of mixtures including a plurality of materials.

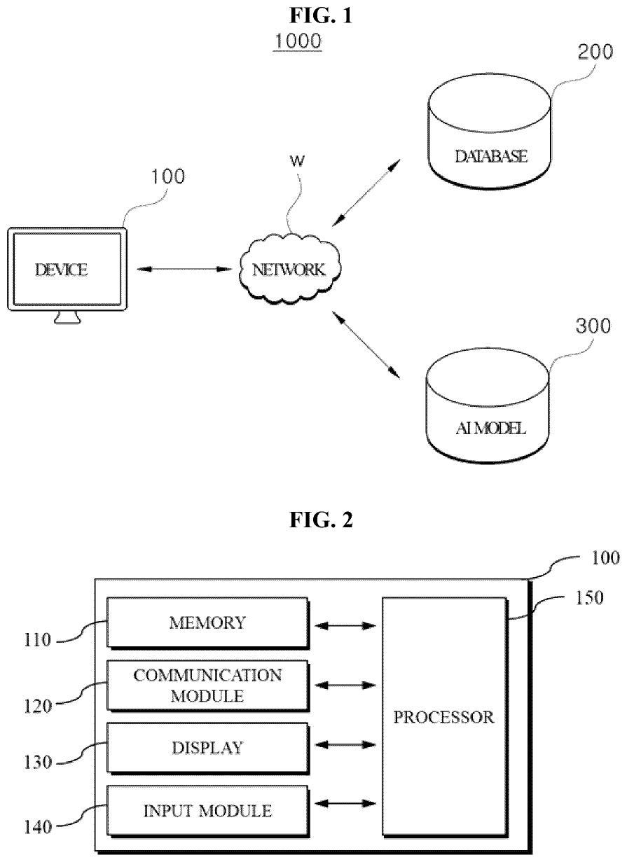

FIG. 1 is a schematic diagram of a system 1000 for implementing a method for predicting physical properties of a mixture including a plurality of component materials according to one embodiment of the present disclosure.

As shown in FIG. 1, a system 1000 for implementing the method of predicting the physical properties of the mixture including the plurality of component materials can include a device 100, a database 200, and an artificial intelligence (AI) model 300.

The device 100, the database 200, and the AI model 300 included in the system 1000 can perform communication via a network W. Here, the network W can include a wired network and a wireless network. For example, the network can include various networks such as a local area network (LAN), a metropolitan area network (MAN), and a wide area network (WAN).

In addition, the network W can include the well-known World Wide Web (WWW). However, the network W according to the present disclosure is not limited to the above-listed networks and can include a well-known wireless data network, a well-known telephone network, or a well-known wired and wireless television network as at least a portion thereof.

The device 100 can predict the physical properties of the mixture including the plurality of materials based on the AI model 300. That is, the device 100 can predict the physical properties of the mixture based on the AI model that can consider interaction between component materials constituting the mixture.

Specifically, the device can obtain first feature data associated with each of the corresponding component materials by inputting material information corresponding to each of the plurality of component materials constituting the mixture into a first AI model.

Here, the first feature data can include molecular feature data (e.g., a molecular feature vector, etc.) of each of the corresponding component materials. The molecular feature data can be based on information obtained from an external device but is not limited thereto and can also be obtained through calculation (e.g., Python program, RdKit, Mordred, etc.).

In certain embodiments, the material information can include chemical information, and the chemical information can include various types of molecular information (e.g., a simplified molecular-input line-entry system (SMILES), an international chemical identifier (INCHI), a self-referencing embedded string (SELFIES)), and the like). As an example, the device can obtain the first feature data associated with each of the corresponding component materials by inputting molecular information of each of the plurality of component materials constituting the mixture into the first AI model.

In other embodiments, the chemical information can include at least one of atom properties and bond properties related to the mixture.

In some embodiments, the device can obtain physical property prediction information associated with the mixture by inputting the first feature data into a second AI model. In some embodiments, the device can obtain the physical property prediction information associated with the mixture by inputting the first feature data and component ratio information of the plurality of component materials in the mixture into the second AI model. Here, the physical property prediction information can include Gibbs free energy prediction information but is not limited thereto.

In some embodiments, the device can predict the physical properties of the mixture based on the obtained physical property prediction information (e.g., the Gibbs free energy prediction information).

As an example, a difference (e.g., a root mean square error (RMSE)) between the Gibbs free energy prediction information obtained by the device and actual Gibbs free energy can be less than 0.02. However, the present disclosure is not limited thereto, and the RMSE value can vary depending on an experimental environment/the number of training times of the model, etc.

The database 200 can store various training data for training the AI model 300. In some embodiments, the database 200 can store the chemical information of two or more materials constituting the mixture. In some embodiments, the database 200 can store the component ratio information of the plurality of component materials in the corresponding mixture.

FIG. 1 shows a case in which the database 200 is implemented outside the device 100. In this case, the database 200 can be connected to the device 100 in a wired or wireless communication manner. However, this is only one embodiment, and the database 200 can also be implemented as one component of the device 100.

The AI model 300 can include a model trained for predicting the physical properties of the mixture including the plurality of materials. Specifically, the AI model 300 can include the first AI model trained to extract the feature data of each component material constituting the mixture and the second AI model trained to extract the physical property prediction information associated with the mixture.

In some embodiments, the first AI model and the second AI model can be trained in an end-to-end manner. The end-to-end training manner is a method of training an AI model to process at once from an input to an output without a pipeline network. Here, the pipeline network is a partial network forming the entire network. That is, when trained in an end-to-end manner, the first AI model and the second AI model may be implemented as a single AI model.

Accordingly, the device can obtain the Gibbs free energy prediction information associated with the mixture by inputting the feature data of each material constituting the mixture into the AI model trained in an end-to-end manner.

However, this is only one embodiment, and the first AI model and the second AI model can be implemented as separate models (or separate pipeline networks) and each model can be trained separately.

FIG. 1 shows a case in which AI model 300 is implemented outside the device 100 (e.g., implemented in a cloud-based manner), but the AI model 300 is not limited thereto, and can be implemented as one component of the device 100.

FIG. 2 is a block diagram for describing a configuration of a device that performs an artificial intelligence-based method for predicting the physical properties of the mixture according to one embodiment of the present disclosure.

As shown in FIG. 2, the device 100 can include a memory 110, a communication module 120, a display 130, an input module 140 and a processor 150. However, the present disclosure is not limited thereto, and software and hardware components of the device 100 can be modified/added/omitted according to a required operation within a scope obvious to those skilled in the art.

The memory 110 can store data supporting various functions of the device 100 and a program for operating the processor 150, store input/output data (e.g., music files, still images, videos, etc.), and store a plurality of application programs or applications that are driven on the present device, and data and commands for operating the device 100. At least some of the application programs can be downloaded from an external server via wireless communication.

Such memory 110 may include at least one type of storage medium among a flash memory type, a hard disk type, a solid state disk type (SSD type), a silicon disk drive type (SDD type), a multimedia card micro type, a card-type memory (e.g., an SD or XD memory), a random access memory (RAM), a static random access memory (SRAM), a read-only memory (ROM), an electrically erasable programmable ROM (EEPROM), a programmable ROM (PROM), a magnetic memory, a magnetic disk and an optical disk.

In some embodiments, the memory 110 can include a database that is separate from the present device but is connected in a wired or wireless communication manner. That is, the database 200 shown in FIG. 1 can be implemented as one component of the memory 110.

The communication module 120 can include one or more components that enable communication with an external device, and can include at least one of, for example, a broadcasting reception module, a wired communication module, a wireless communication module, a short-range communication moduleand a location information module.

The wired communication module can include not only various wired communication modules such as a LAN module, a WAN module and a value added network (VAN) module, but also various cable communication modules such as a universal serial bus (USB), a high definition multimedia interface (HDMI), a digital visual interface (DVI), a recommended standard 232 (RS-232), power line communication and a plain old telephone service (POTS).

In addition to a WiFi module and a wireless broadband (WiBro) module, the wireless communication module can include a wireless communication module for supporting various wireless communication methods such as global system for mobile communication (GSM), code division multiple access (CDMA), wideband CDMA (WCDMA), universal mobile telecommunications system (UMTS), time division multiple access (TDMA), long term evolution (LTE), 4G, 5G, or 6G.

The display 130 displays (outputs) information (e.g., the feature data output from each AI model/the physical property prediction information (e.g., the Gibbs free energy prediction information)/physical property prediction result information of the mixture, etc.) processed by the device 100.

For example, the display can display execution screen information of an application program (e.g., an application) driven on the device 100, or user interface (UI) or graphic user interface (GUI) information according to such execution screen information. The type of UI output by the display 130 will be described below.

The input module 140 is for receiving information from a user, and when receiving information through a user input unit, the processor 150 can control the operation of the device 100 to correspond to the input information.

The input module 140 can include a hardware physical key (e.g., a button located on at least one of a front surface, a back surface, and a side surface of the present device, a dome switch, a jog wheel, a jog switch, etc.) and a software touch key. In some embodiments, the touch key can be formed as a virtual key, a soft key, or a visual key that is displayed on a touchscreen embodiment of the display 130 through software processing, or the touch key can be a physical touch key disposed on a portion of input module 140 other than a touchscreen. The virtual key or visual key can have various forms, can be displayed on a touchscreen and can be formed as, for example, a graphic, a text, an icon, a video, or a combination thereof.

The processor 150 can be implemented with a memory that stores data for an algorithm for controlling the operations of the components in the device 100 or a program that reproduces the algorithm, and at least one processor (not shown) that performs the above-described operation using the data stored in the memory. In this case, each of the memory and the processor can be implemented as a separate chip. Alternatively, the memory and the processor can also be implemented as a single chip.

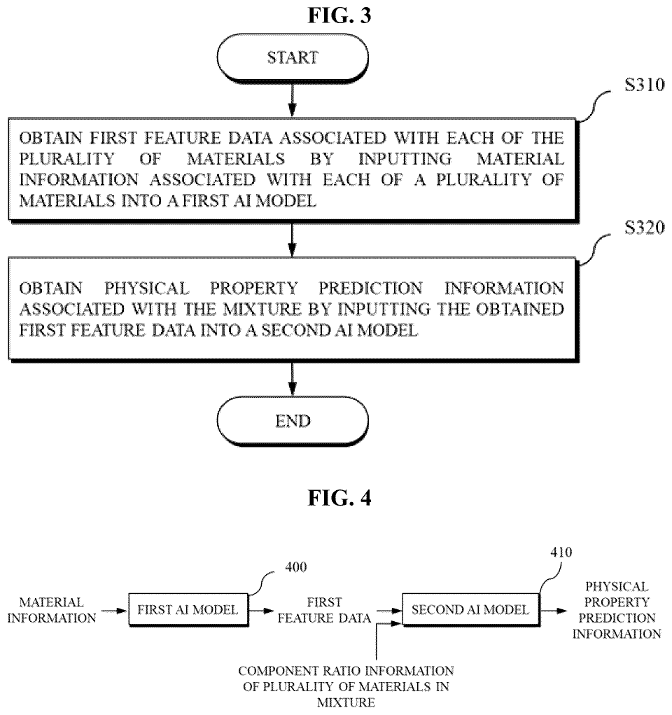

In some embodiments, the processor 150 can control the operations of the components in the device 100 by combining any one or a plurality of the above-described components in order to implement various embodiments according to the present disclosure, which embodiments will be described in FIGS. 3 to 8 below.

FIG. 3 is a block diagram illustrating an artificial intelligence-based method for predicting the physical properties of the mixture according to one embodiment of the present disclosure.

The device can obtain first feature data associated with each of the plurality of component materials (S310) by inputting material information of each of a plurality of component materials into a first AI model.

Here, the material information can include chemical information. In certain embodiments, the chemical information can include molecular information (e.g., a SMILES, an INCHI, or a SELFIES) of each of the plurality of component materials.

The first AI model can include a model trained to output the first feature data (e.g., molecular feature data of each of the materials, etc.) of each of the corresponding materials. As shown in FIG. 4, input data of the first AI model 400 can include the material information of each of the plurality of materials constituting the mixture, and output data of the first AI model 400 can include the first feature data.

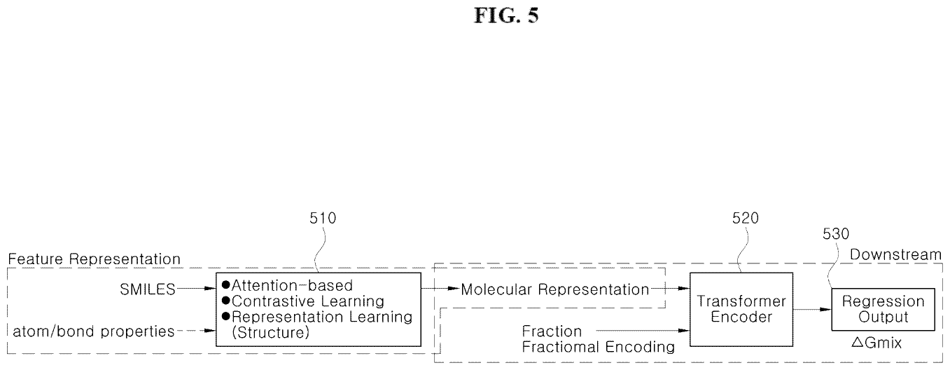

For example, as shown in FIG. 5, the first AI model 500 may include at least one of an attention-based model, a molecular contrastive learning-based modeland a representative learning-based model.

Here, the attention-based model can be trained to extract feature data of chemical information for a specific component material based on physicochemical properties (e.g., at least one of polarizability and hydrophobicity (e.g., sLogP)) of the specific component material. That is, the attention-based model can be trained to extract the first feature data from each specific component material in turn after training based on the specific physical property.

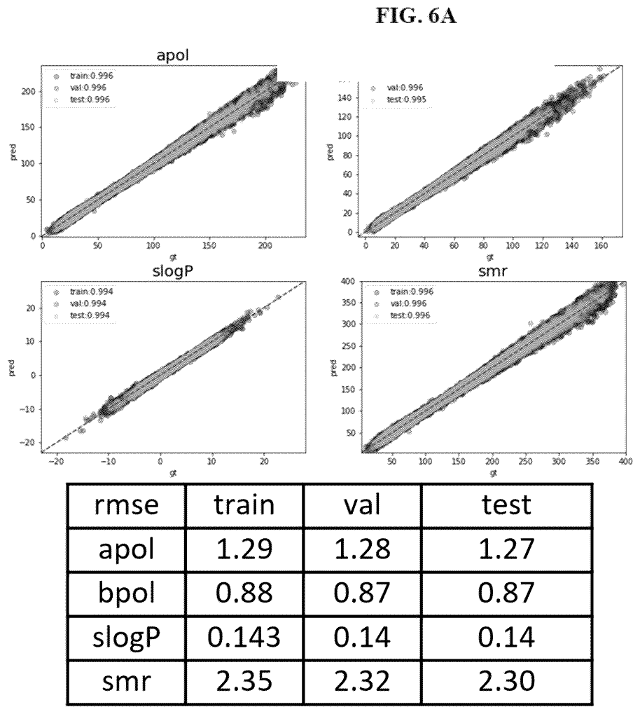

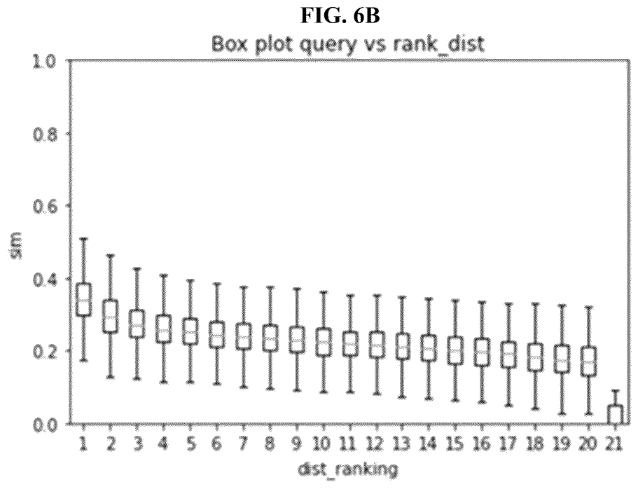

FIG. 6A shows an example of the result data of training of the attention-based model. The attention-based model can be trained to extract the feature data of the chemical information for the specific material based on information that is highly relevant to the Gibbs free energy (e.g., a Wildman-Crippen LogP property (sLogP), a Wildman-Crippen molar refractivity (MR) property (smr), a bond polarizability property (bpol), or an atomic polarizability property (apol), etc.).

The molecular contrastive learning-based model can be trained to align molecules with similar structures on a latent space. That is, the molecular contrastive learning-based model can be trained to calculate how many molecules with similar structures exist nearby and to align molecules with similar structures based on the result of the calculation. The latent space is a space of feature data (or molecular feature vector) of chemical information of a specific material.

For example, FIG. 6B shows an example of the result data of training of the molecular contrastive learning-based model. The x-axis of the graph shown in FIG. 6B indicates similarity between molecules, and the y-axis indicates a distance. According to the graph shown in FIG. 6B, as the distance between molecules increases, the similarity between molecules tends to decrease.

As another example, the first AI model can be trained to update second feature data extracted from two-dimensional graph information of a specific material based on third feature data extracted from three-dimensional structural characteristics (e.g., a plurality of spatial arrangement conformers for the specific material) of the specific material. In addition, the first AI model can be trained to output the first feature data based on the updated data. In this case, the first AI model can be trained to consider the plurality of spatial arrangement conformers for the specific material.

Specifically, when the chemical information implemented as the two-dimensional graph information for the specific material is input, the pre-trained first AI model can process the second feature data extracted from the corresponding chemical information to be updated by reflecting the third feature data extracted from the three-dimensional structural characteristics for the specific material. Accordingly, the second feature data can be updated to include the three-dimensional structural characteristics. In addition, the pre-trained first AI model can output the first feature data based on the updated data.

As another example of the present disclosure, the chemical information that is one example of the material information can include at least one of atom properties and bond properties related to the mixture. For example, the device can obtain the first feature data by inputting, into the first AI model, the molecular information (e.g., a SMILES, an INCHI, or a SELFIES) of each of the plurality of materials. That is, the first AI model can be trained to output the first feature data based on the molecular information related to the mixture (considering both atom properties and bond properties).

Only the molecular information can be input into the first AI model, but the present disclosure is not limited thereto, and at least one of the atom properties and the bond properties can be selectively input.

The device can obtain physical property prediction information associated with the mixture (S320) by inputting the obtained first feature data into a second AI model.

Specifically, the device can obtain the physical property prediction information associated with the mixture by inputting the first feature data and component ratio information of the plurality of materials in the mixture into the second AI model. Here, the component ratio information (or fraction information) of the plurality of component materials in the mixture can be information encoded in a predefined manner.

That is, as shown in FIG. 4, input data of the second AI model 410 can include at least one of the first feature data or the component ratio information of the plurality of materials. In addition, output data of the second AI model 410 can include the component ratio information of the plurality of component materials in the mixture.

In addition, the second AI model can be trained to output the physical property prediction information for the mixture (e.g., Gibbs free energy prediction information) based on the component ratio information of the plurality of component materials in the mixture and the first feature data.

As an example, the second AI model can include at least one of a multi-layer perceptron (MLP)-based model, a ResNet model and a transformer encoder model. The device can obtain prediction information associated with the physical properties of the mixture based on the physical property prediction information (e.g., the Gibbs free energy prediction information).

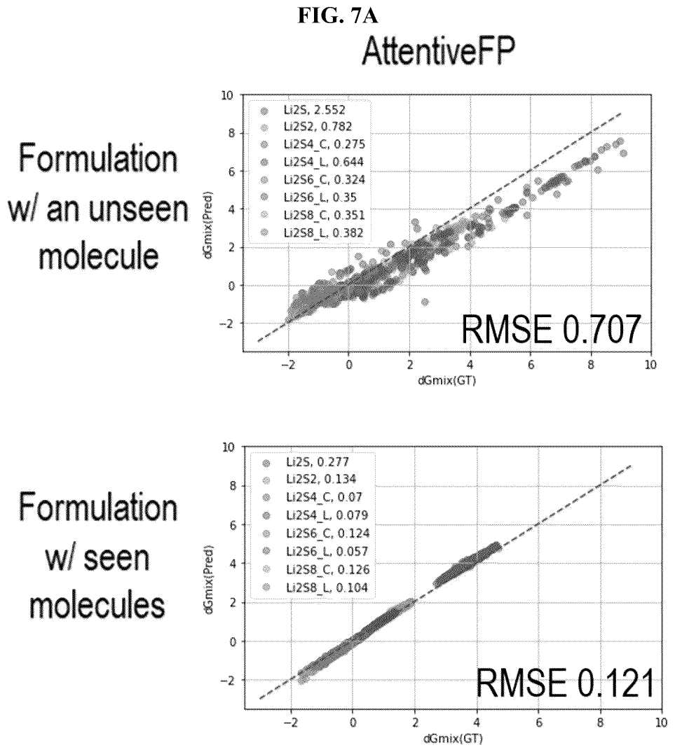

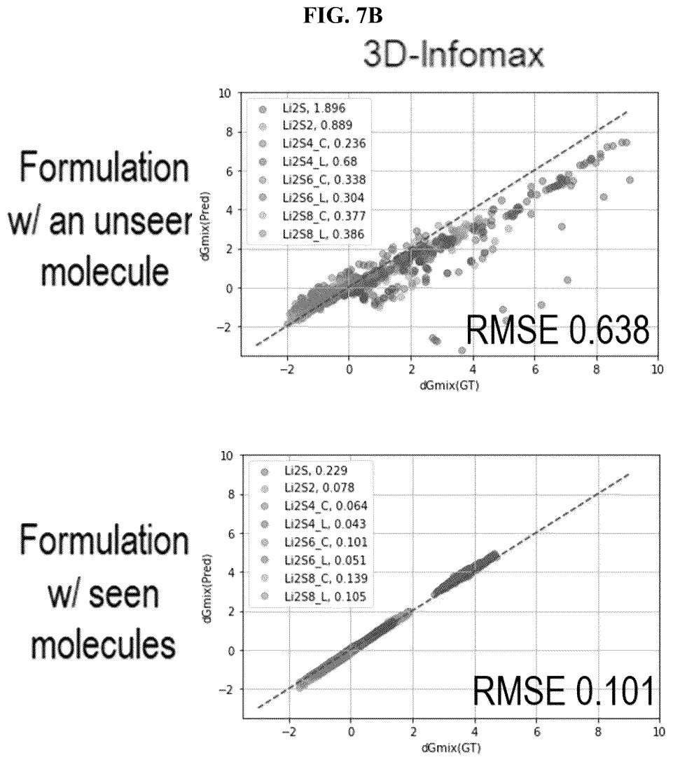

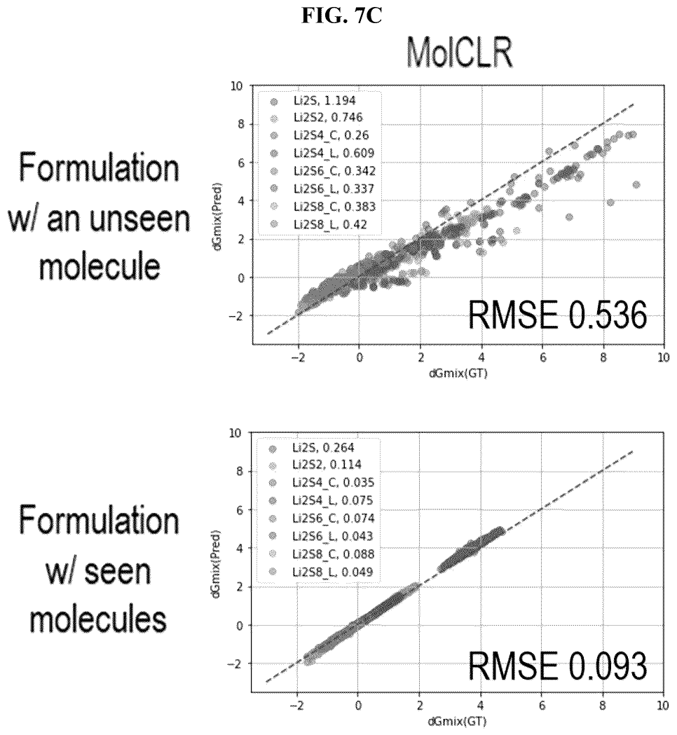

FIGS. 7A, 7B, and 7C each are diagrams for describing type-specific performance of the AI models according to one embodiment of the present disclosure.

FIG. 7A shows an example of the result of extracting the feature data according to the first AI model when the first AI model includes the attention-based model.

FIG. 7B shows an example of the result of extracting the feature data according to the first AI model when the first AI model is trained to update the second feature data extracted from the two-dimensional graph information of the specific material based on the third feature data extracted from the three-dimensional structural characteristics of the specific material.

FIG. 7C shows an example of the result of extracting the feature data according to the first AI model when the first AI model includes the molecular contrastive learning-based model.

The upper image of each of FIGS. 7A, 7B, and 7C shows the result of extracting the feature data for the mixture, which was not a training target during the training process, by the first AI model. The lower image of each of FIGS. 7A, 7B, and 7C shows the result of extracting the feature data for the mixture, which was the training target during the training process, by the first AI model.

When comparing the upper and lower images of each of FIGS. 7A, 7B, and 7C, it can be confirmed that an RMSE value decreases in a case in which the first AI model extracts the feature data for the mixture that was the training target during the training process compared to the other case where the mixture was not the training target of the first AI model.

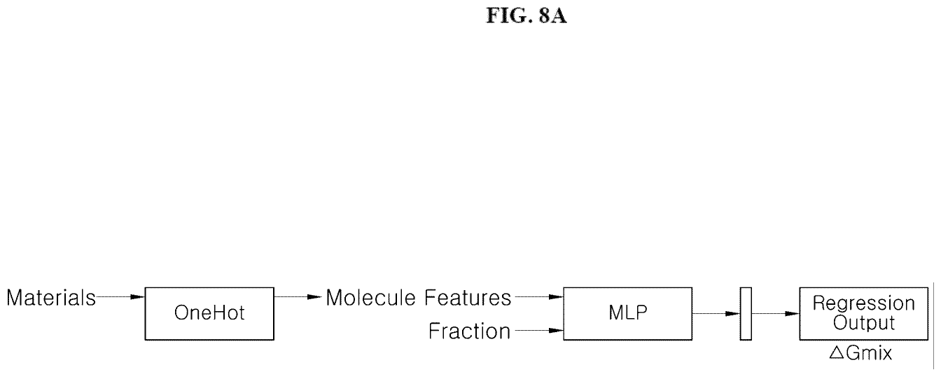

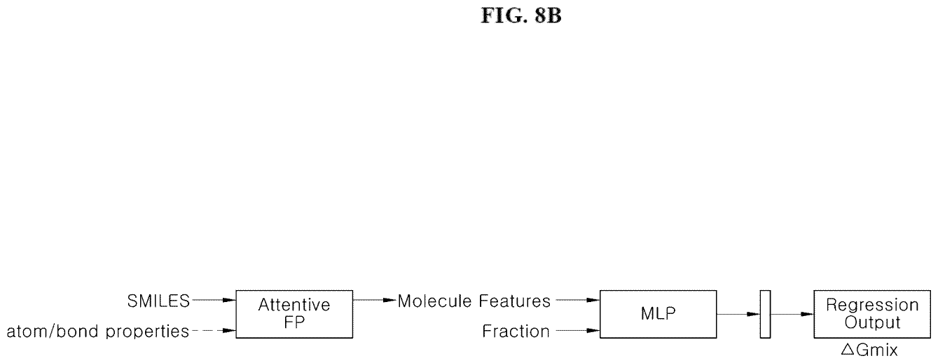

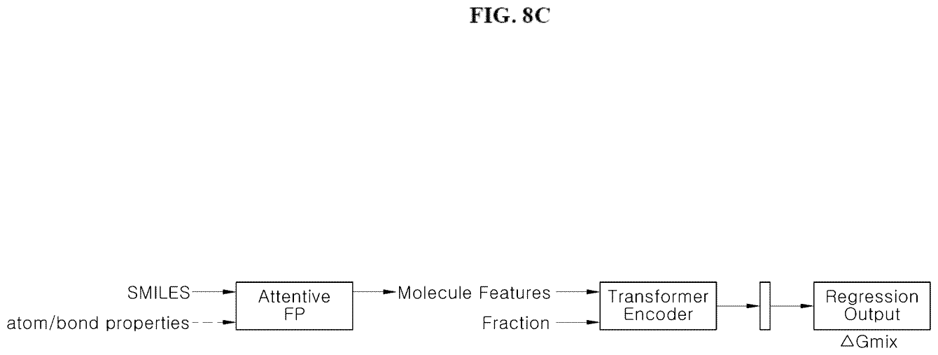

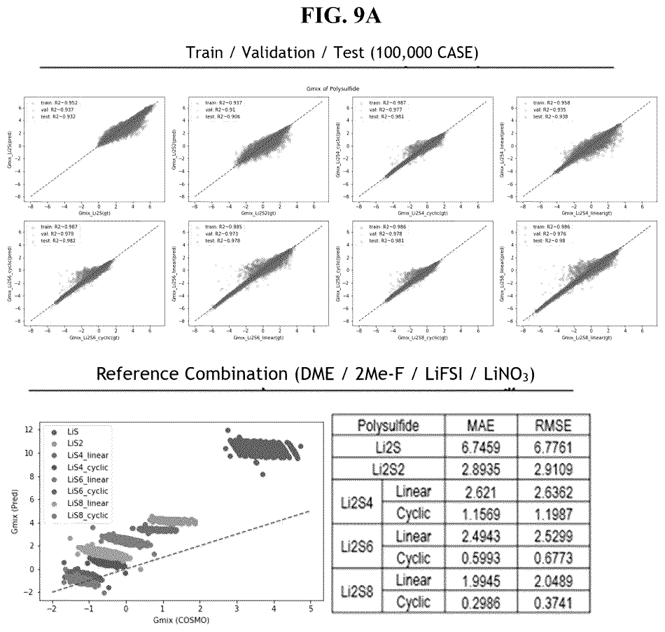

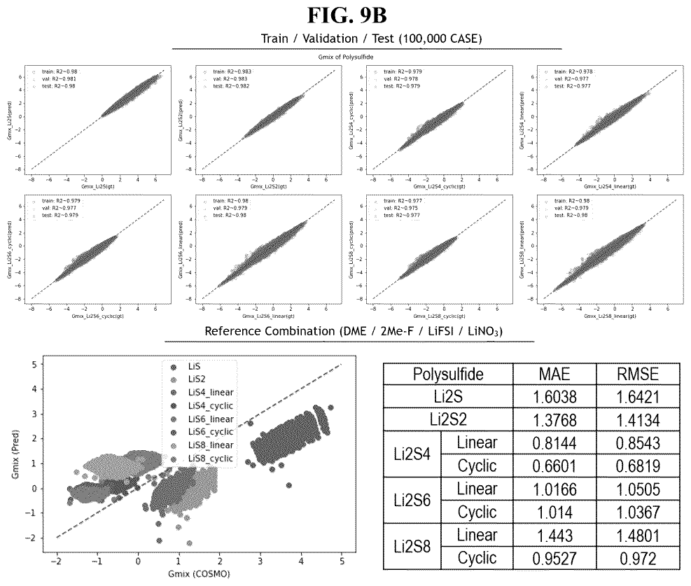

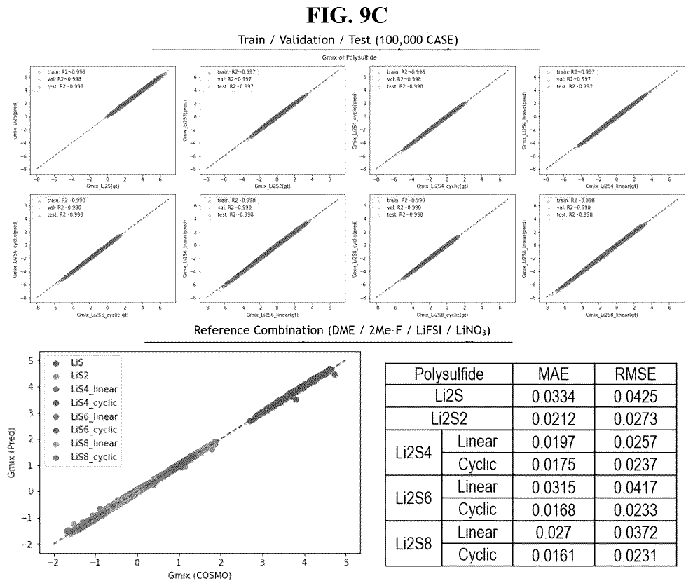

FIGS. 8A, 8B, and 8C are diagrams illustrating structures of the AI models for predicting the physical properties of the mixture according to one embodiment of the present disclosure. FIGS. 9A, 9B, and 9C are diagrams illustrating training/inference methods of the AI models for predicting the physical properties of the mixture according to an embodiment of the present disclosure.

In FIGS. 8B and 8C, cases in which the first AI model is implemented as an attentive FP model (i.e., an example of an attention-based model) are shown, but the present disclosure is not limited thereto. The first AI model can be implemented as an AI model that can extract feature data of a material, such as the molecular contrastive learning-based model.

In addition, in FIGS. 8B and 8C, the operations of inputting, into the first AI model, the molecular information (e.g., a SMILES, an INCHI, or a SELFIES) as the material information of the material constituting the mixture are shown, but the present disclosure is not limited thereto. The input data for the first AI model can be composed of only the material information.

In some embodiments, at least one of the atom properties or the bond properties for the mixture can be selectively input into the first AI model. In other embodiments, at least one of the atom properties and the bond properties for the mixture can be obtained based on the molecular information.

In FIGS. 8A, 8B, and 8C, the operations in which the second AI model (e.g., the transformer encoder) outputs the Gibbs free energy prediction information are shown, but the present disclosure is not limited thereto and the second AI model may output various types of the physical property prediction information.

FIG. 8A shows a process of extracting the physical property prediction information associated with the mixture through a one hot encoding model and the MLP-based model. In FIG. 8B, when the material information of each of the plurality of materials constituting the mixture is input, the one hot encoding model may be trained to output molecular feature data mapped to the input material information. In this case, the one hot encoding model may be trained to output information pre-mapped to the chemical information, rather than extracting the feature data from the material information (e.g., the chemical information) of the materials constituting the mixture.

FIG. 8C shows a process of extracting the physical property prediction information (e.g., the Gibbs free energy prediction information) associated with the mixture through the first AI model including the attention-based model and the second AI model based on the MLP-based model. FIG. 8C also shows a process of extracting the physical property prediction information (e.g., the Gibbs free energy prediction information) associated with the mixture through the first AI model including the attention-based model and the transformer encoder model.

As described above, unlike FIGS. 8B and 8C, FIG. 8A does not include a process of extracting the first feature data associated with each of the plurality of component materials constituting the mixture through the first AI model.

FIGS. 9A, 9B, and 9C show result data according to FIGS. 8A, 8B, and 8C.

When comparing FIGS. 9A with 9B, the performance difference that occurs due to utilizing the feature data extracted from the molecular information (e.g., a SMILES, an INCHI, or a SELFIES) of the component materials constituting the mixture can be confirmed. When utilizing the feature data extracted from the material information of the component materials, difference values between actual physical property information (e.g., Gibbs free energy) and the physical property prediction information (e.g., predicted Gibbs free energy information) obtained through the AI model may decrease.

When comparing FIGS. 9A with 9B, the performance difference between the MLP-based model and the transformer encoder model can be confirmed. When utilizing the transformer encoder model, more accurate prediction information can be output than when utilizing the MLP-based model.

Some disclosed embodiments may be implemented in the form of a recording medium in which computer-executable commands are stored. The commands can be stored in the form of program code, and, when executed by a processor, program modules are generated to perform operations of the disclosed embodiments. The recording medium may be implemented as a computer-readable recording medium.

The computer-readable recording medium available to skilled practitioners includes all types of recording media in which computer-decodable commands are stored. For example, these can include a ROM, a RAM, a magnetic tape, a magnetic disk, a flash memory, an optical data storage device, and the like.

As described above, the disclosed embodiments have been described with reference to the accompanying drawings. Those skilled in the art to which the present disclosure pertains will understand that the present disclosure may be implemented in different forms from the disclosed embodiments without departing from the technical spirit or essential features of the present disclosure. The disclosed embodiments are illustrative and should not be construed as being limited.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A device for predicting physical properties of a mixture including a plurality of component materials, the device comprising:
a memory storing:
a first artificial intelligence (AI) model trained to output first feature data associated with material information corresponding to specific component materials and
a second AI model trained to output mixture physical property prediction information associated with the first feature data; and
a processor configured to execute the first AI model and the second AI model,
wherein the processor is configured to:
obtain first feature data associated with each of the plurality of component materials by inputting, into the first AI model, material information corresponding to each of the plurality of component materials; and
obtain mixture physical property prediction information by inputting the first feature data into the second AI model.

2. The device of claim 1, wherein the first AI model includes an attention-based model, and
the attention-based model is trained to extract feature data of a specific component material based on at least one of polarizability and hydrophobicity of the specific component material.

3. The device of claim 1, wherein the first AI model includes a molecular contrastive learning-based model, and
the molecular contrastive learning-based model is trained to align molecules with similar structures on a latent space.

4. The device of claim 1, wherein the first AI model is trained to update second feature data extracted from two-dimensional graph information of a specific component material based on third feature data extracted from three-dimensional structural characteristics of the specific component material, and is trained to output the first feature data based on the updated data.

5. The device of claim 4, wherein the three-dimensional structural characteristics of the specific component material include a plurality of spatial arrangement conformers of the specific component material.

6. The device of claim 1, wherein the second AI model includes a multi-layer perceptron-based model.

7. The device of claim 1, wherein the second AI model includes a transformer encoder model.

8. The device of claim 1, wherein the processor is configured to:
obtain physical property prediction information associated with the mixture by inputting the first feature data and component ratio information of the plurality of component materials in the mixture into the second AI model.

9. The device of claim 1, wherein the first AI model and the second AI model are trained in an end-to-end training manner.

10. The device of claim 1, wherein the material information includes chemical information,
the chemical information includes molecular information of each of the plurality of component materials, and
the molecular information includes at least one of a simplified molecular-input line-entry system (SMILES), an international chemical identifier (INCHI), or a self-referencing embedded string (SELFIES).

11. The device of claim 10, wherein the chemical information includes at least one of atom properties and bond properties related to the mixture.

12. The device of claim 1, wherein the first feature data includes molecular feature data of each of the plurality of component materials.

13. The device of claim 1, wherein the physical property prediction information includes Gibbs free energy prediction information.

14. A method for predicting physical properties of a mixture including a plurality of component materials, performed by a device, the method comprising:
obtaining first feature data associated with each of the plurality of component materials by inputting material information associated with each of the plurality of component materials into a first AI model that has trained first feature data associated with material information; and
obtaining physical property prediction information associated with the mixture by inputting the first feature data into a second AI model that has trained physical property prediction information associated with the first feature data.

15. A computer program that is stored in a computer-readable recording medium coupled with a hardware device to execute the method for predicting the physical properties of the mixture including the plurality of component materials of claim 14.

16. The method of claim 14, wherein the material information is at least one of a simplified molecular-input line-entry system (SMILES), an international chemical identifier (INCHI), or a self-referencing embedded string (SELFIES).

17. The method of claim 14, wherein the material information is a polarizability or a hydrophobicity.
